(19) United States
(12) Patent Application Publication
(10) Pub. No.: US 2019/0159804 A1
(43) Pub. Date: May 30, 2019

(54) Medical Needle System

(71) Applicant: Blaine S. Cameron, Plantation, FL (US)

(72) Inventor: Blaine S. Cameron, Plantation, FL (US)

(21) Appl. No.: 15/822,964

(22) Filed: Nov. 27, 2017

(51) Int. Cl. A61B 17/34; A61M 25/06; A61M 5/32

(52) U.S. Cl. CPC A61B 17/3401; A61M 5/46; A61M 5/3286; A61M 25/0606

(57) ABSTRACT

A spinal needle that includes a needle body insertable through tissue to a desired depth within a patient's body, the spinal needle is capable of retaining bodily fluids, including cerebrospinal fluid, and provides a window capable of allowing a user to view the existence or non-existence of bodily fluid retained therein.

### FIELD OF THE INVENTION

The present invention relates to a spinal needle, and more particularly to a spinal needle constructed to reduce incidence of complications in intrathecal procedures.

### BACKGROUND OF THE INVENTION

Spinal needles have the benefit of supplying a dense neural block that inhibits nerve receptors and provides highly effective pain relief. However, there are certain negative side effects that occur following a puncture to the dura.

One well known complication following spinal anesthesia and other spinal needles is post spinal puncture headache (“PSPH”). PSPH occurs due to dural and arachnoid puncture from a loss of cerebrospinal fluid.

Currently, following puncture of the dura such that the catheter is threaded into the epidural space, the spinal needle is removed. However, during said removal, there is a period in which the cerebrospinal fluid is able to escape prior to the insertion of the catheter. Further, during the transition between the spinal needle and the catheter, there is a high incidence of cerebrospinal fluid escape.

PSPH incidence can be as high as 36% during ambulatory diagnostic lumbar punctures causing onset of PSPH with onset starting immediately upon loss of cerebrospinal fluid to a week later.

Spinal anesthesia is one of the most frequently employed methods of regional anesthesia. This regional anesthesia is accomplished by the temporary interruption of nerve transmission using a local anesthetic injected into the readily identifiable subarachnoid space. The ensuing anesthesia is predictable, occurs rapidly, and is associated with profound muscle relaxation. The patient may be wide awake, or if preferred, the anesthetic may be supplemented with varying amounts of sedative-tranquilizers. Spinal anesthesia is particularly useful for surgery involving the lower extremities, pelvis, perineum, and lower abdomen.

The spinal column, which surrounds the spinal cord, is formed by a series of vertebrae separated by cartilaginous intervertebral disks and united by a series of ligaments. The body of each vertebra bears the weight of the patient and forms the base of the neural arch. The arch, which surrounds the spinal cord, is made up of a pedicle and lamina on each side. Between the laminae of each vertebra there is a posterior opening in the vertebral canal. It is through this opening that a spinal needle is passed when performing a subarachnoid block.

In adults the spinal cord varies in length from 40 to 45 cm and ends at various levels of the vertebral column depending on the age of the patient. In newborns, the spinal cord extends to the third lumbar vertebra, but in the adult it usually ends at the lower border of the first lumbar vertebra because the spinal cord does not grow as much as the vertebral column. Thirty-one pairs of symmetrically arranged spinal nerves are each attached to the spinal cord by an anterior and posterior root. Because the spinal cord is shorter than the vertebral column, the spinal cord segments in adults do not lie opposite their corresponding vertebrae. The spinal nerve roots must travel obliquely in a caudad direction to reach their respective intervertebral foramina. The roots of the lumbar, sacral, and coccygeal nerves comprise the cauda equina and are necessarily the largest and longest in order to reach their intervertebral foramen. The greater size of these nerve roots provides a larger surface area to be exposed to the action of local anesthetics, thus allowing more rapid onset of anesthesia.

The spinal cord is covered by three membranes or meninges. The dura mater (the outermost membrane) is the downward continuation of the meningeal layer of the cranial dura mater. The middle of the three coverings, the arachnoid is a thin membrane closely adherent to the dura mater. The dura and the arachnoid are in close contact that usually it is not possible to puncture the dura without also piercing the arachnoid. Nevertheless, on rare occasions, the tip of the conventional epidural or spinal needle may accidentally enter the subdural space. Local anesthetic inadvertently injected into the subdural space will diffuse poorly and result in inadequate contact with the nerve roots. Poor or absent anesthesia may ensue. Should subdural placement occur during an attempted epidural anesthetic, the improper position of the needle may not be recognized and the injection of an epidural dose of local anesthetic may result in a much higher block than anticipated.

Delivering spinal anesthesia may be accomplished using a lumbar puncture procedure. The lumbar puncture generally involves a spinal needle 9 cm long. The spinal needle is usually adequate for lumbar puncture, but longer ones (10-15 cm) are available for the occasional obese patient or difficult paramedian approach. The removable, close-fitting stylet helps stiffen the needle and prevents coring of the tissue. Commonly, two sizes of spinal needles are used, 22-27 gauge. The larger diameter 22 gauge needle is easier to direct and renders the characteristic feel of the various ligaments penetrated easier to appreciate. However, the incidence of postspinal headache is increased with the larger needle, particularly if the larger needle is also equipped with a standard point which is a cutting bevel.

A postdural puncture headache is the most common postoperative complication of spinal anesthesia. The incidence increases with the larger spinal needles and those with a cutting bevel at the tip but decreases with increasing patient age. Postdural puncture headache also occurs more commonly in women than in men, and more often in pregnant women than in nonpregnant women. The headache is positional in that it comes on in the upright position and is relieved or at least improved in the recumbent position.

The causative mechanism of the postdural puncture headache is believed to be associated with the continuing leakage of cerebrospinal fluid (CSF) through the dural opening left by the spinal needle. The leakage of CSF causes a decrease in CSF pressure which, in turn, produces compensatory cerebral vasodilation. Bringing the patient into the erect position also results in traction on the pain-sensitive, dilated blood vessels. Accordingly, conservative therapy for the postdural puncture headache consists of bed rest and analgesics.

There is currently no adequate spinal needle system or device capable of sufficiently reducing the loss of cerebrospinal fluid escape following the transition between the spinal needle and the catheter. Nor is there an adequate needle for indicating cerebral spinal fluid contained within the body of the needle.

There is a need in the art for an improved spinal needle system.

### SUMMARY OF THE INVENTION

The present invention advantageously provides a spinal needle for delivering spinal anesthesia with reduced loss of spinal fluid.

According to an embodiment of the present invention, a principal object is to provides a spinal needle with a one-way valve for preventing loss of spinal fluid during the transition from the removal of a stylet that is then replaced by the injection spinal anesthesia from the needle body and delivered to a patient's spine.

According to an embodiment of the present invention, another object is to provide a spinal needle capable of reaching the posterior opening in the vertebral canal for delivering spinal anesthesia and indicating via cerebral spinal fluid that the needle is properly positioned within a patient's body.

According to an embodiment of the present invention, another object is to provide a hub that will enable cerebrospinal fluid flash back, ph confirmation, and filter to remove particulate matter and glass from injectate.

Yet another object of the present embodiment is to provide a needle that reduces cerebrospinal fluid loss.

Yet another object of the present embodiment is to provide a hub that will be enclosed and contain a ph indicator and filter on the end.

According to an embodiment of the present invention, another object is to provide visually confirmation portion of the needle body for indicating that cerebrospinal fluid is contained within the needle body, which is accomplished by a clear or opaque portion that allows the user/doctor to view inside at least a portion of the needle body. This viewable portion may optionally contain an indicator, such as a ph indicator, contained with the needle body. The indicator contacts cerebrospinal fluid within the needle body for visually indicating the existence of cerebrospinal fluid. The visual confirmation portion, such as a window, and/or the indicator allows the user/doctor to confirm that the spinal needle is positioned properly inside the patient.

According to yet another non-limiting embodiment of the present invention, another object is to provide a delivery device that includes three components, including a sharp, hollow introducer component a few centimeters in length that is used to puncture the skin, a more blunt hollow needle component several centimeters in length that is slideably disposed within the hollow introducer to allow the caregiver to delicately pierce the dura membrane, and a stylet component that is slideably disposed within the needle to selectively occlude the needle and control the flow of fluid therein. The introducer and needle components both have hubs on their proximate ends. The hubs act as handles or grips to facilitate manipulation of the introducer and needle.

According to one embodiment of the present embodiment, a principal object is to provide a medical needle system an introducer that has an introducer hub, an introducer cannula that has a first introducer cannula end and a second introducer cannula end, the introducer cannula has a hollow channel between the first and second introducer cannula ends, the introducer hub is at least partially surrounded by said first introducer cannula end, a medical needle has a medical needle hub, a medical needle cannula has a first needle end and a second needle end, the medical needle hub at least partially surrounds the first needle end, the medical needle has a needle channel with a bore on the second needle end, the medical needle cannula is capable of removable slidable insertion at least partially through the hollow channel of the introducer, a stylet has a stylet hub and a stylet shaft, the stylet shaft has a first stylet end and a second stylet end, the first stylet end engages the stylet hub, the stylet is capable of removably slidably engaging and disengaging within the medical needle cannula, the stylet shaft when engaged with the medical needle cannula is capable of obstructing entry of fluid through said bore, a retaining body horizontally aligned with the medical needle cannula, the retaining body includes a window capable of allowing a user to view any bodily fluid contents retained therein, a one-way fluid valve disposed about the medical needle at a rear of said retaining body in alignment with the medical needle cannula, a detection member disposed within said retaining body, the detection member is viewable from said window, the one-way fluid valve substantially prevents release of fluids about the medical needle when the stylet is disengaged with the medical needle, and the detection member changes color when in contact with bodily fluid.

Other objects will become evident as the present invention is described in detail below.

### BRIEF DESCRIPTION OF THE DRAWINGS

Embodiments of the present application are described herein in which similar elements are given similar reference characters, and a more complete understanding of the present invention, and the attendant advantages and features thereof, will be more readily understood by reference to the following detailed description when considered in conjunction with the accompanying drawings wherein:

FIG. 1 is a perspective view of a spinal needle with accompanying introducer and stylet in accordance with one embodiment of the present invention;

FIG. 2 is a perspective view of a spinal needle with accompanying introducer and having the stylet removed in accordance with the principles of one embodiment;

FIG. 3 is a side view of a spinal needle separated from the introducer in accordance with the principles of one embodiment;

FIG. 4 is a side view of an introducer in accordance with one embodiment of the present invention; and

FIG. 5 is a side view of a stylet in accordance with one embodiment of the present invention.

### DETAILED DESCRIPTION OF THE INVENTION

The present invention advantageously provides a spinal needle constructed to reduce incidence of complications in intrathecal procedures. The present invention contemplates various types of spinal needle bevels, including without limitation Whitacre needles, Sprotte needles, Quincke needles, and Atraucan needles.

Accordingly, the needle components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

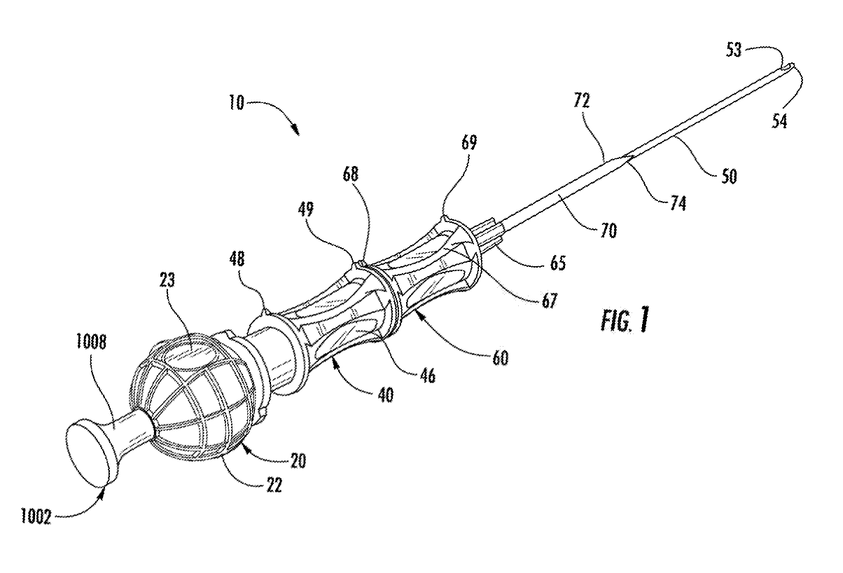

Referring now to the drawings figures in which like reference designators refer to like elements. FIG. 1 shows an exemplary spinal needle constructed in accordance with the principles of the present invention and designated generally as needle 10. In particular, needle 10 includes a needle body 40. The needle body 40 includes cannula 50 with a pointed distal tip 54. The pointed distal tip 54 is insertable through tissue to a desired depth within a patient's body.

The needle body 40 further includes a hub 46 that is disposed to the upper end of the cannula 50, the hub 46 extending from an upper hub portion 48 to a lower hub portion 49. A bulb portion 20 is disposed about an upper hub portion 48. The bulb portion 20 includes a retaining body 22 capable of retaining bodily fluids, including cerebrospinal fluid. The retaining body 22 includes a window 23 capable of allowing a user to view the existence or non-existence of bodily fluid retained therein.

In one non-limiting embodiment, the bulb retaining body 22 is constructed to receive cerebrospinal fluid that enters through a bore 53, allowing a person to view the cerebrospinal fluid through the window 23.

In one-non-limiting embodiment, the inside of the window 23 is coated with a cerebrospinal fluid detection substance that indicates upon contact with cerebrospinal fluid. Such indication may include changing colors on the inside of the window 23.

In one non-limiting embodiment, the hub 46 is opaque to allow visual indication of cerebrospinal fluid contained therein.

In one-non-limiting embodiment, internal to the bulb portion is material that visually indicates to a user/doctor of the existence of bodily fluids, including cerebrospinal fluid. For example, a color changing indicator may be provided within the hub 46 for reacting to bodily fluids.

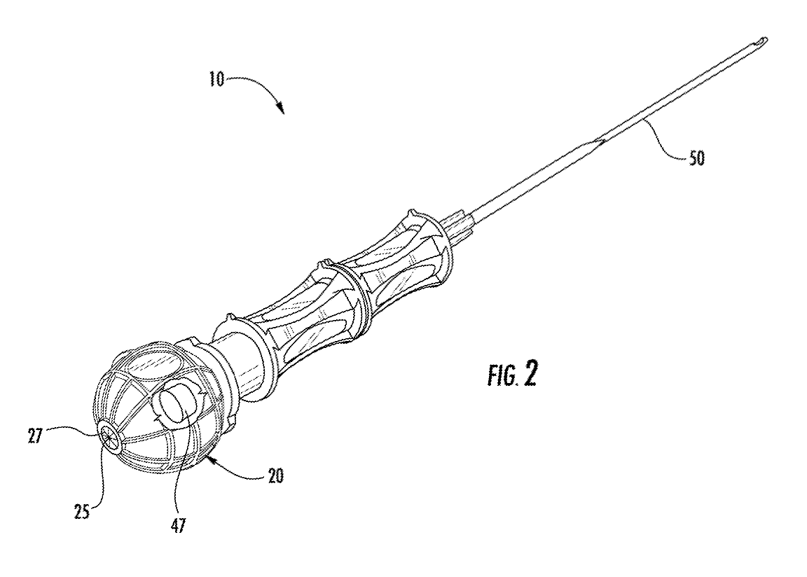

In one-non-limiting embodiment, internal to the hub 46 is an indicator member 47 that visually indicates to a user/doctor of the existence of bodily fluids, including cerebrospinal fluid. For example, a color changing indicator member 47 may be provided within the hub 46 for reacting to bodily fluids (FIG. 2).

Referring to FIG. 2, the bulb portion 20 of the spinal needle 10 includes an aperture 25 in vertical alignment with the cannula 50. The aperture 25 is provided for allowing insertion and removal of a stylet 1002 (the stylet 1002 shown in inserted with the needle body 40 in FIG. 1 and isolated from the needle body 40 in FIG. 5).

In one non-limiting embodiment, the spinal needle 10 is shown with bulb portion 20 and having the stylet 1002 (FIG. 5) removed from aperture 25.

In one non-limiting embodiment, aperture 25 is fitted with a one way valve 27 that permits the insertion and removal of the stylet 1002, but prevents the exit of bodily fluid, including cerebrospinal fluid, through the aperture 25, therein containing any cerebrospinal fluid within the bulb 20, and preventing continued loss of fluid from a patient's body when, for example, the stylet is removed from the needle body 40.

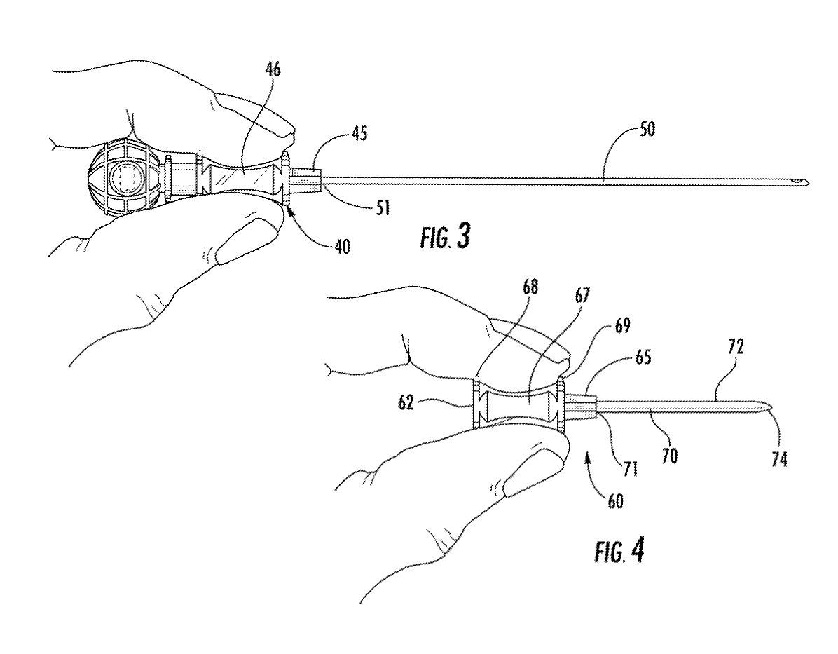

Referring now to FIG. 3, the needle body 40 is shown with hub 46. The cannula 50 engages with the hub 46 via a hub tip 45, the tip 45 surrounding a portion of the proximal portion 51 of the cannula 50 (FIG. 3).

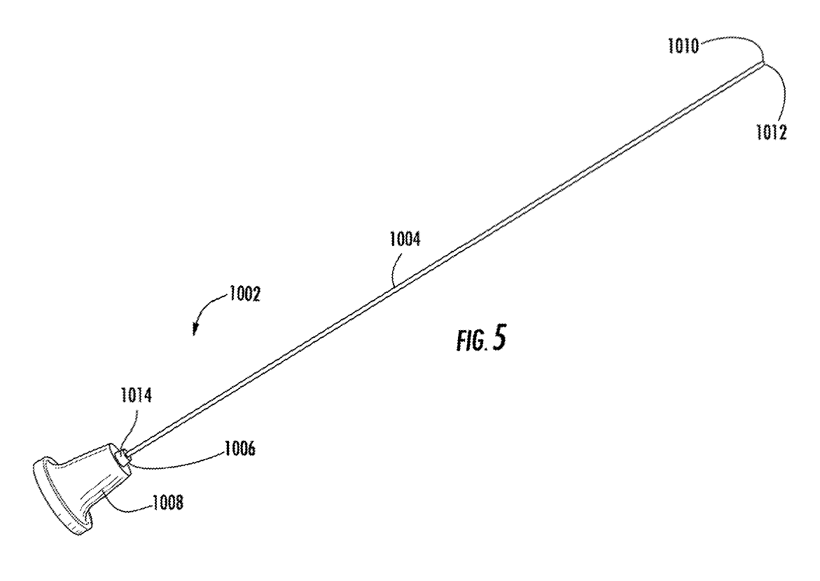

Referring now to FIGS. 1 and 4, the introducer 60 is shown in isolation. The introducer 60 includes introducer hub 67, the introducer hub 67 extending from an upper introducer hub portion 68 to a lower introducer hub portion 69. The introducer hub 67 includes the introducer hub tip 65. The hub tip 65 surrounds a portion of the introducer cannula 70 at a proximal portion 71 maintaining a portion of the cannula 70 thereof. The introducer cannula extends to a distal portion 72 of the introducer point 74. The introducer point 74 is formed with an opening for providing a conduit, the conduit extending between the introducer point 74 and the upper edge 62, the conduit provided for allowing the cannula 50 to pass at least partially therethrough.

Referring now to FIG. 5 is a perspective view of a stylet 1002. The stylet 1002 includes a stylet shaft 1004 connected at a proximal end 1006 with a stylet hub 1008, the stylet hub 1008 is constructed and arranged to allow for gripping by a person.

In one non-limiting embodiment, the stylet hub 1008 (FIG. 1) includes bulb plug 1014 that plugs aperture 25 (FIG. 2) when the stylet 1002 is engaged with the needle body 40.

Still referring to FIG. 5, the stylet is constructed such that the stylet shaft 1004 is placed within the hollow cannula 50 to convert the hollow cannula 50 into a solid insertable needle capable of preventing epidermis, dermis, or other bodily members from entering the cannula 50 (FIG. 3) through the bore 53 (FIG. 1) during insertion of the spinal needle 10 into a patient.

The cannula 50 (FIG. 3) is slightly larger in circumference than a stylet shaft 1004 (FIG. 5) that allows the stylet shaft 1004 to snuggly insert inside a hollow portion of the cannula 50 (FIG. 1 shows the snug fit).

Referring again to FIG. 2, in one non-limiting embodiment, the bulb 20 contains a detection/indication member 47 that provides a visual indication of positive or negative indications of contact with cerebrospinal fluid of the patient.

Referring again to FIG. 5 and in one non-limiting embodiment shown, a stylet tip 1010 is provided with a detection member 1012 that is capable of detecting cerebrospinal fluid of a patient, such that when the stylet shaft 1004 is removed from the cannula 50, the detection member 1012 provides a visual indication of positive or negative indications of contact with cerebrospinal fluid of the patient.

In one non-limiting embodiment, the detection member includes a pH detection substance for visually identifying positive and/or negative contact with cerebrospinal fluid at or near the stylet tip 1010.

In one non-limiting embodiment, the one way valve 27 may be optionally disposed at any location of the needle body between the aperture 25 and the hub tip 45. In this embodiment, the detection member that provides a visual indication of positive or negative indications of contact with cerebrospinal fluid of the patient is positioned before the one way valve 27 towards the distal tip 54 for facilitating contact with the cerebrospinal fluid.

As used herein, when the term “and/or” is used, it shall include all combinations of one or more of the associated described items.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are contemplated in light of the above teachings without departing from the scope and spirit of the invention. It will be readily apparent that various changes may be made thereto without departing from the spirit and scope of the disclosure or sacrificing all of its material advantages. The examples and embodiments described herein are merely exemplary of the instant disclosure.

What is claimed is:

1. A medical needle system comprising:
an introducer having an introducer hub, an introducer cannula having a first introducer cannula end and a second introducer cannula end, said introducer cannula having a hollow channel between said first and second introducer cannula ends, said introducer hub at least partially surrounding said first introducer cannula end;
a medical needle having a medical needle hub, a medical needle cannula having a first needle end and a second needle end, said medical needle hub at least partially surrounding said first needle end, said medical needle having a needle channel with a bore on said second needle end, said medical needle cannula capable of removable slidable insertion at least partially through said hollow channel of said introducer;
a stylet having a stylet hub and a stylet shaft, said stylet shaft having a first stylet end and a second stylet end, said first stylet end engaging said stylet hub, said stylet capable of removable slidable engages and disengages within said medical needle cannula, said stylet shaft when engaged with said medical needle cannula is capable of obstructing entry of fluid through said bore;
a one-way fluid valve disposed about said medical needle; and
wherein said one-way fluid valve substantially prevents release of fluids about said medical needle when said stylet is disengaged with said medical needle.

2. The medical needle system of claim 1, wherein said medical needle hub further includes a retaining body horizontally aligned with said medical needle cannula.

3. The medical needle system of claim 2, wherein said retaining body includes a window capable of allowing a user to view any bodily fluid contents retained therein.

4. The medical needle system of claim 3, wherein said one-way fluid valve is disposed at a rear of said retaining body in alignment with said medical needle cannula.

5. The medical needle system of claim 4, further including a detection member disposed within said retaining body.

6. The medical needle system of claim 5, wherein said detection member is viewable from said window.

7. The medical needle system of claim 6, wherein said detection member changes color when in contact with bodily fluid.

8. The medical needle system of claim 6, wherein said detection member changes color when in contact with cerebrospinal fluid.

9. The medical needle system of claim 1, further including a detection member disposed about said second stylet end.

10. The medical needle system of claim 9, wherein said detection member changes color when in contact with cerebrospinal fluid.

11. A spinal needle system comprising:
an introducer having an introducer hub, an introducer cannula having a first introducer cannula end and a second introducer cannula end, said introducer cannula having a hollow channel between said first and second introducer cannula ends, said introducer hub at least partially surrounding said first introducer cannula end;
a medical needle having a medical needle hub, a medical needle cannula having a first needle end and a second needle end, said medical needle hub at least partially surrounding said first needle end, said medical needle having a needle channel with a bore on said second needle end, said medical needle cannula capable of removable slidable insertion at least partially through said hollow channel of said introducer;
a stylet having a stylet hub and a stylet shaft, said stylet shaft having a first stylet end and a second stylet end, said first stylet end engaging said stylet hub, said stylet capable of removable slidable engages and disengages within said medical needle cannula, said stylet shaft when engaged with said medical needle cannula is capable of obstructing entry of fluid through said bore;
a retaining body horizontally aligned with said medical needle cannula, said retaining body includes a window capable of allowing a user to view any bodily fluid contents retained therein;
a one-way fluid valve disposed about said medical needle at a rear of said retaining body in alignment with said medical needle cannula;
a detection member disposed within said retaining body, said detection member is viewable from said window;
wherein said one-way fluid valve substantially prevents release of fluids about said medical needle when said stylet is disengaged with said medical needle; and
wherein said detection member changes color when in contact with bodily fluid.
